(19) United States
(12) Patent Application Publication
(10) Pub. No.: US 2026/0224619 A1
(43) Pub. Date: Aug. 6, 2026

(54) T CELL ADOPTIVE TRANSFER IN LYMPHOREPLETE HOSTS MEDIATED BY TRANSIENT STAT5B ACTIVATION DURING ENGRAFTMENT

(71) Applicant: MUSC FOUNDATION FOR RESEARCH DEVELOPMENT, Charleston, SC (US)

(72) Inventors: Richard ONEIL, Charleston, SC (US); Megan TENNANT, Charleston, SC (US)

(21) Appl. No.: 19/147,777

(22) PCT Filed: Nov. 7, 2023
(86) PCT No.: PCT/US2023/036907

§ 371 Date: Jul. 14, 2025

Related U.S. Application Data
(60) Provisional application No. 63/440,728, filed on Jan. 24, 2023.

(51) Int. Cl. A61K 35/17; A61K 40/11; A61K 40/42; A61P 35/00; C07K 14/435; C12N 5/00; C12N 5/0783; C12N 15/85

(52) U.S. Cl. CPC A61K 35/17; A61K 40/11; A61K 40/42; A61P 35/00; C07K 14/435; C12N 5/0636; C12N 5/562; C12N 15/85; C12N 2510/00

(57) ABSTRACT

The present invention relates to methods for promoting functional engraftment of engineered T cells that would obviate the need for lymphodepletion.

### PRIORITY TO RELATED APPLICATIONS

This application claims the benefit of U.S. Provisional Application No. 63/440,728, filed Jan. 24, 2023. The entire contents of the above-identified application is hereby incorporated by reference in its entirety.

### STATEMENT REGARDING FEDERALLY SPONSORED RESEARCH OR DEVELOPMENT

This invention was partially made with Government support under NCI P30CA138313. The Government has certain rights in the invention.

### FIELD OF THE INVENTION

The invention relates generally to methods for promoting functional engraftment of engineered T cells that would obviate the need for lymphodepletion. All publications, patents, patent applications, and other references cited in this application are incorporated herein by reference in their entirety for all purposes and to the same extent as if each individual publication, patent, patent application or other reference was specifically and individually indicated to be incorporated by reference in its entirety for all purposes. Citation of a reference herein shall not be construed as an admission that such is prior art to the present invention.

### BACKGROUND OF THE INVENTION

The success of adoptive cell therapy (ACT) using tumor-infiltrating lymphocytes (TILs), chimeric antigen receptor (CAR) or T cell receptor (TCR) engineered T cells has relied upon preconditioning regimens designed to lymphodeplete patients prior to infusion (1, 2). Lymphodepleting regimens such as chemotherapy and total body irradiation (TBI eliminate endogenous cytokine-responsive cells that act as cytokine sinks (3). Further, lymphodepletion eradicates suppressive cell populations within the host immune system by eliminating immunosuppressive cell types including regulatory T cells (4, 5) and myeloid-derived suppressor cells (MDSCs) (6). Patients who undergo lymph depleting regimens prior to ACT enjoy better response rates and improved survival and antitumor immunity (1, 7). However, while these regimens are critical for maximizing clinical benefit, the agents used like fludarabine and cyclophosphamide are largely nonspecific agents with significant toxicity profiles (8, 9).

Lymphodepleting regimens can induce a variety of side-effects including leukopenia, pulmonary veno-occlusive disease and reactive myelopoiesis (10). These side effects have contributed to patient exclusion and hindered wider adoption of clinical trials leveraging engineered T cell-based therapeutics. In addition to the significant side effects, the physiological response to lymphodepletion can adversely affect the efficacy of ACT in qualifying patients (11). Further, management of these toxicities often necessitate long-term inpatient support and hospitalization, causing extreme financial burden on both patients and healthcare providers. Despite the fact that lymphodepletion regimens carry many drawbacks, it is clear that they promote functional engraftment and persistence of transferred T cells (12, 13), while ACT without prior lymphodepletion inhibits engraftment of adoptively transferred T cells (14) and reduces clinical benefit.

A need exists in the art for cell autonomous methods for promoting functional engraftment of engineered T cells that would obviate the need for lymphodepletion.

### SUMMARY OF THE INVENTION

The present invention is directed to a method for functional engraftment of cells in the absence of lymphodepletion, comprising the step of transient activation of Stat5 signaling by transfection of T cells with a plamid or RNA encoding a constitutively active Stat5 molecule or a constitutively active cytokine receptor that activates STAT5 signalling, Stat3, Stat5a, Stat5b, Interleukin 7 receptor, Interleukin 2 Receptor, Interleukin 9 Receptor, Interleukin 15 Receptor and engineered orthogonal receptors prior to adoptive transfer.

The present invention is also directed to a method for reducing cytokine release syndrome (CRS) in a patient undergoing cancer therapy, comprising the step of administering T cells transiently transfected with an RNA or DNA encoding an activated form of Stat5 or cytokine receptor prior to adoptive transfer.

The present invention is further directed to a method for increasing efficiency of engraftment of cells, comprising the step of transient activation of Stat5 signalling by transfection of DNA or RNA encoding an activated Stat5 or cytokine receptor prior to adoptive transfer.

Additionally, the present invention is directed to a method for reducing production of IL-6 in a patient in need thereof, comprising the step of administering to said patient transient activation of Stat5 signalling by transfection of DNA or RNA encoding an activated Stat5 or cytokine receptor prior to adoptive transfer.

### BRIEF DESCRIPTION OF THE DRAWINGS

FIG. 1 shows that transient expression of Stat5b* promotes CD8⁺T cell engraftment in the absence of lymphodepletion. a) Experimental schematic demonstrating three groups of animals that underwent adoptive transfer (ACT) either using the conventional 5 gy lymphodepletion, no lymphodepletion followed by ACT, no lymphodepletion followed by ACT with cells that have been transfected with a plasmid encoding STAT5* immediately prior to ACT. b) Representative ventral images of ffLuc-Thy 1.1 engineered CD8⁺T cells 3 days after ACT. c) Animals were measured for 29 days post ACT and the intensity of the signal emitted by the ffLuc-Thy 1.1+T cells was expressed as luminescence. Quantification of total (dorsal +ventral) luminescence is plotted (n=10 for LD, and LD pT-CMV-mStat5b*, n=5 no LD and Stat5b* mRNA). ACT using Stat5* yields significantly more efficient engraftment when comparing to the conventional method of lymphodepletion prior to ACT. d) Peripheral cytokine levels 24 hours prior to ACT and 4 days post-ACT. Shown are cytokines thought to be involved in ACT associated toxicities in humans. *p<0.05 by unpaired Student's t-test (d). Error bars indicate the S.E.M.

FIG. 2 shows that Stat5 drives superior recall response in the absence of lymphodepletion. a) Experimental schema for b-c. b) Representative images of ffLuc-Thy1.1 engineered CD8⁺T cells 40 days post-ACT and 24 hours after rechallenging with LLC-ova cells. c) Animals were measured 24- and 72 -hours following LLC-ova challenge and the intensity of the signal emitted by the ffLuc-Thy 1.1 transfected T cells was expressed as luminescence. d) Tumor growth kinetics and e) survival of mice challenged with LLC-ova

FIG. 3 shows that CD62L re-expression is reduced by transient expression of Stat5*A) schematic diagram of experiment, mice were either transferred using conventional lymphdepletion or by transient expression of Stat5*. B and C) representative plots and graphical summary of flow cytometry data comparing CD62L expression and F) is a schematic diagram of a competition study examining if the effects of lymphodepletion on CD62L expression are dominant over those of transient Stat5* expression during engraftment. G and E) representative plots and summary of data comparing phenotype of GFP+ and thy 1.1+ (Stat5* transfected) cells from the same spleens

FIG. 4 shows that ATC without lymphodepletion using transient Stat5* results a unique phenotype of tumor infiltrating T cell. A) Schematic diagram of experiments: subcutaneous tumors were initiated prior to ACT. Tumors were harvested 6 days after adoptive transfer and the phenotype of engrafted cells was assessed by flow cytometry for markers associated with T cell exhaustion. B), C) PD1 levels are suppressed, while PD1+Tim3+double positive cells were unchanged due to a strong induction of Tim3 in Stat5* engrafted cells despite no changes in Lag-3 expression

FIG. 5 shows that adoptive transfer using transient Stat5b* without lymphodepletion leads to functional control of tumors. a) Experimental schema for b-e. b) Tumor growth kinetics and c) survival of mice bearing LLC-ova tumors infused with OT1⁺T cells engrafted via conventional lymphodepletion (blue) or Stat5b* (red) (n=5 mice per group). d) Tumor growth kinetics and e) survival of mice bearing B16-F10 tumors infused with PMEL⁺T cells engrafted via conventional lymphodepletion (blue) or stat5b* (red) (n=5 mice per group). *p<0.05, **p<0.01, ***p<0.001, ****p<0.0001 by linear regression of tumor growth and log-rank, mantel-cox test of survival proportions (c, e). Error bars indicate the S.E.M.

FIG. 6 shows that cryopreserved human T cell products retain and express transfected mRNA after extended cryo-storage.

FIG. 7 shows that cryopreserved Stat5b* mRNA modified T cells are highly functional and efficiently engraft in lymphoreplete mice.

### DETAILED DESCRIPTION OF THE INVENTION

It is to be understood that the descriptions of the present invention have been simplified to illustrate elements that are relevant for a clear understanding of the present invention, while eliminating, for the purpose of clarity, many other elements found in typical pharmaceutical compositions. Those of ordinary skill in the art will recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art. Furthermore, the embodiments identified and illustrated herein are for exemplary purposes only, and are not meant to be exclusive or limited in their description of the present invention.

Functional engraftment is characterized by expansion and persistence of T lymphocytes and anti-tumor function. The specific mechanisms that contribute to effective engraftment are thought to be related to homeostatic T cell proliferation. The increased availability of homeostatic cytokines that occurs after lymphodepletion leads to an enhanced state of T cell proliferation thought to be regulated through γ_{c} cytokines including interlukin-2 (IL-2), IL-7 and IL-15. These cytokines play pivotal roles in regulating T cell homeostasis and inflammatory responses through critical signaling pathways. Current lymphodepleting regimens eliminate cellular cytokine sinks, thus increasing the availability of homeostatic cytokines to support the effectiveness of adoptively transferred T cells (3).

Conventionally, the cytokines IL-2, IL-7, and IL-15 signal through the JAK-STAT pathway, primarily through the activation of JAK1/3 and STAT5, known mediators of T-cell anti-tumor immunity (15, 16). In response to cytokine stimulation, the C-terminal tyrosine (mY699, hY694) of Stat5b becomes phosphorylated, mediating Stat5b activation and dimerization, thus facilitating nuclear translocation and DNA binding (17, 18). The inventors chose to utilize a form of Stat5b* which mimics structural aspects of phosphorylated Stat5b independently of tyrosine phosphorylation by substituting histidine 298 for arginine which is referred to as Sta5b* in this manuscript.

The inventors believe that transient Stat5b activation during the initial phase of adoptive transfer obviates the need for prior lymphodepletion. The inventors describe that transient expression of an active form of Stat5 during the initial phase of adoptive transfer leads to functional T cell engraftment and eliminates the spike in IL-6 typically observed during engraftment, which is a driving factor for cytokine release syndrome (CRS). The inventors observed that cells engrafted in lymphoreplete mice using Stat5b* have superior recall response and improved tumor control in multiple immune competent tumor models. These results show that this novel engraftment strategy may represent an opportunity to remove lymphodepletion regimens from ACT protocols, relying instead on transient Stat5b* expression during the engraftment phase and also improve T cell function after ACT.

Thus, the inventors discovered that transient expression of a constitutively active STAT5 (STAT5-CA) during adoptive transfer obviates the need for prior lymphodepletion, leads to long term functional engraftment, and improves tumor control in immune competent cancer models.

In one embodiment, the inventors discovered that transient transfection of CAR-T cells with a constitutively active STAT5 immediately before adoptive transfer allows for functional engraftment of cells in the absence of lymphodepletion.

In one embodiment of the invention, the inventors have found that the transient expression (via DNA, mRNA, cirRNA) of constitutively active receptors that activate signaling pathways like Jak/Stat, and other common gamma chain cytokine receptors, facilitate T cell engraftment and function. This is based on the demonstration that cell autonomous adoptive transfer of functional T cells uses a method that does not require genetic modification of the T cells or lymphodepletion of the host. This rationally extends to other constitutively active receptors that can be transiently delivered to T cells to safely enhance adoptive transfer without the risk for transformation. This also extends to use of this transient expression methodology for enhancing adoptive transfer or T cell function in the context of mild, reduced, or conventional lymphodepletion.

These constitutively active molecules can include but are not limited to constitutively active forms of Stat3, Stat5a, Stat5b, Interleukin 7 receptor, Interleukin 2 Receptor, Interleukin 9 Receptor, Interleukin 15 Receptor and engineered orthogonal receptors,

In another embodiment of the invention, the inventors discovered that adoptive transfer of T cells without prior lymphodepletion results in reduced production of several cytokines including IL6. IL6 production is characteristically identified as antagonistic to therapeutic utility by driving cytokine release syndrome (CRS) and contributing to T cell dysfunction [2].

In a further embodiment of the invention, the inventors discovered that transient transfection of T cells with STAT5-CA prior to adoptive transfer increases the efficiency of engraftment such that much lower doses of cells can be administered thereby reducing product manufacturing time and further reducing the risk of side effects.

In another embodiment of the present invention, provided is a cryopreserved T cell product ready for infusion without host lymphodepletion. The T cell manufacturing process requires harvesting T cells from patients by leukapheresis, activation and lentiviral transduction of cells with a CAR expressing construct, and expansion of the transduced cells for 2-3 weeks. After manufacture, the cells are cryopreserved and shipped to the point-of-care where they are thawed and infused.

The success of adoptive cell therapy using tumor-infiltrating lymphocytes (TILs), CAR, or T cell receptor (TCR) engineered T cells relies on efficient engraftment of the cellular product. Historically, preconditioning regimens designed to lymphodeplete patients prior to infusion have been used to facilitate engraftment of adoptively transferred cells. Lymphodepletion (LD) is now considered essential for effective CAR-T therapy and is a universal component of adoptive T cell transfer protocols. However, while these regimens have historically thought to be critical for maximizing clinical benefit, the agents used (fludarabine and cyclophosphamide), are largely nonspecific agents with significant toxicity profiles. Replacing, simplifying, or improving the LD step has been a major priority for pharmaceutical companies over the past decade. The inventors have developed an approach that relies on transfection of manufactured T cells with an mRNA that encodes a constitutively active mutein of Stat5b (Stat5b*) prior to adoptive transfer. The inventors observe that this transient activation of the Stat5b pathway ‘tricks’ the T cells into thinking that they are entering a lymphodepleted host and results in efficient functional engraftment of the T cell product that turns out to be superior to conventional lymphodepletion based protocols. This technology could obviate the need for LD and revolutionize T cell therapy.

### EXAMPLES

The following examples further describe and demonstrate particular embodiments within the scope of the present invention. Techniques and formulations generally are found in Remington's Pharmaceutical Sciences (Mack Publishing Co., Easton, Pa.). The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### General Materials and Methods

### Molecular Biology

Transfection-grade plasmids were prepared according to the manufacturers protocol using endotoxin-free buffers and ZymoPURE II Plasmid Maxiprep Kits (ZYMO Research, USA) The plasmid vectors pT-effluc-thy1.1 20 and pRP--CAG>hyPBase 29, pTPB-CMV-GFP, pCMV-mStat5b*, pT7-mStat5b*, and pT7-hStat5b* were synthesized by Vector Builder Biosciences (Chicago, IL). All plasmid vectors were confirmed by DNA sequencing. Transfection-grade plasmids were prepared according to the manufacturers protocol using endotoxin-free buffers and ZymoPURE II Plasmid Maxiprep Kits (ZYMO Research, USA).

### Mice

OT1 (C57BL/6-Tg(TcraTerb)1100Mjb/J), PMEL (B6.Cg-Thyla/Cy Tg(TcraTcrb)8Rest/J) and B6 albino (B6(Cg)-Tyrc-2J/J) mice were obtained from the Jackson Laboratory. Luciferase (β-actin-luc) were obtained from Taconic Bioscience. All animal experiments were approved by the Medical University of South Carolina (MUSC) Institutional Animal Care and Use Committee and the Division of Laboratory Animal Resources at MUSC maintained all mice.

### Cell Culture

B16-F10 and LLC-OVA tumor lines, as well as HEK293T cells, were maintained in Dulbecco's Modified Eagle's Medium (DMEM) supplemented with 10% FBS. All cell lines were determined to be mycoplasma-free in December 2021. OT-1 T cells, human CD4+ T cells, and human CD8+ T cells were maintained in RPMI supplemented with 10% FBS, 300 mg/L L-glutamine, 100 units/mL penicillin, 100 μg/mL streptomycin, 1 mM sodium pyruvate, 100 μM NEAA, 1 mM HEPES and 55 μM 2-mercaptoethanol . For OT-1 and PMEL T-cell activation and expansion, whole splenocytes from OT1 or PMEL mice were activated with 1 μg/mL OVA 257-264 peptide or 1 μg/mL glycoprotein 100 (gp100). For Luc+ T cell isolation, CD8+ lymphocytes were purified from the spleen using the MACS mouse CD8a+ T-cell isolation Kit (Miltenyi Biotec, Auburn, CA). Purified CD8+ lymphocytes were then activated with anti-CD3e 2.5 μg/ml (BD Bioscience, San Jose, CA) and anti-CD28 2 μg/ml (BD Bioscience, San Jose, CA). All T cells were expanded for 3 days with 200 U/mL rhIL-2 (NCI). T cells were split on day 3 and expanded in rhIL-2 or IL-15 (50ng/ml) through day 7.

### In Vitro Transcription

mRNA was transcribed using the mMessage mMachine T7 ULTRA Transcription Kit (Ambion, Life Technologies, Grand Island, NY). The protocol provide by the manufacturer was used. The mRNA was resuspended in nuclease free water and stored at −80° C. prior to transfection.

### Adoptive Transfer Models

After in vitro expansion, lymphocytes cells were washed briefly in PBS by centrifugation and transfected with the Neon (Life Technologies, Grand Island, NY) transfection system according to the manufacturer's instructions for mouse T cells. In vitro-expanded OT-1 T cells were prepared for adoptive transfer by transfection with 5 μg pRP--CAG>hyPBase and 10 μg of pCMV-EL-Thy1.1 or 10 μg of pT-GFP and 10 μg of pCMV-mStat5b*. For mRNA transfections, OT-1 T cells were transfected with the indicated concentrations of GFP mRNA or with 20 μg of Stat5b* mRNA using the previously described settings 30. Transfected T cells were rested in complete T cell media for 1 hour before resuspending cells in PBS and directly transferring the cells in to recipient mice. Luc+ T cells were transfected with 20 μg Stat5b* mRNA. Prior to adoptive transfer, mice were preconditioned by exposure to 5 Gy of lymphodepleting radiation using a cesium irradiator or using Cyclophosphamide (Thermo Scientific, Waltham, MA) dissolved in PBS administered by intraperitoneal injection at a dose of 200 mg/kg unless otherwise indicated. T cells were transfected as described above, allowed to recover in complete TCM for 1 h and then adoptively transferred via IP injection. For ex vivo analysis of ELThy1.1 or GFP transferred T cells, mice were sacrificed 3 days after transfer and organs were processed to single-cell suspensions for FACS analysis.

### Tumor Cell Challenge

For primary challenges, mice were injected subcutaneously (s.c.) with 2.5×105 LLC-ova tumor cells on day 40 after initial adoptive transfer of OT-1+ T cells.

### Tumor Models

For adoptive cellular therapy experiments, LLC-ova adenocarcinomas or B16-F10 melanomas were established subcutaneously by injecting 2.5×105 cells into the right flank of male B6(Cg)-Tyrc-2J/J mice and tumor-bearing hosts were irradiated with 5 Gy 24 hours prior to T-cell transfer. After 7 days of tumor growth, 1×107 OT1 or PMEL T cells were transfected using the Neon transfection system prior to infusion in 100 μL phosphate-buffered saline via IP injection into mice. Tumor growth was measured every other day with calipers, and survival was monitored with an experimental endpoint of tumor growth >400 mm2. For ex vivo analysis of ff-Luc-Thy1.1 transferred T cells, mice were sacrificed 5 days after transfer and organs were processed to single-cell suspensions for FACS analysis.

### Bioluminescent Imaging

Mice were anesthetized using isoflurane and injected IP with luciferin substrate (Perkin Elmer, Waltham, MA) at standard concentration of 150 mg/kg in PBS. Approximately 10 minutes after luciferase injections, mice were imaged on the AMI-HT (Spectral Imaging, Tuscon AZ). All data shown represent mean luminescence observed by summing dorsal and ventral measurements obtained from identical regions of interest drawn over the trunk and head of each individual mouse.

### Assessment of Serum Cytokines

Blood was collected in Microvette CD300 Potassium EDTA collection tubes (Sarstedt, Newton, NC) by retroorbital vein bleeding. Serum was measured using the Mouse Cytokine Proinflammatory Focused 10-Plex Discovery Assay (MDF10) (Eve Biotechnologies, Calgary AB, Canada).

### Flow Cytometry

Cells were stained with Live/Dead Fixable Aqua dead stain cell kit (Invitrogen) prior to extracellular staining. Fluorescent dye-conjugated antibodies were purchased from Biolegend (San Diego, CA), eBioscience (San Diego, CA) or BD Pharmigen (Mountain View, CA). Cells were stained extracellularly CD90.1(HIS51)-PE, CD8a (53-6.7)-PerCP-eFluor 710, CD44 (IM7)-PECy7, CD62L (MEL-14)-BV421, PD-1 (29F.1A12)-APC, Tim-3 (RMT3-23)-FITC, Lag-3 (C9B7W)-APC-eFluor 780, CD 3(145 -2C11)-FITC, (GK 1.5)-APC-eFluor 780 and CD25 (PC61.5)-PECy7 or isotype control and subsequently. Cell surface antibody staining was done in PBS containing 2% FBS. Antibody-stained cells were run directly on a CytoFLEX flow cytometer (Beckman Coulter Life Sciences, Indianapolis, IN) and analysis was performed with FlowJo software.

### Nanostring Gene Expression Analysis

RNA isolation: Cells were immediately pelleted by centrifugation at 4° C. and resuspended in Homogenization Buffer containing thioglycerol provided in the purification kit. RNA was extracted with a Promega Maxwell RSC 16 using the Maxwell RSC simplyRNA Cells kit (Cat# AS1390) according to the manufacturer's instructions and RNA concentration was measured using a NanoDrop 8000.

Gene Quantitation: Direct quantitation of selected transcripts was performed using 100 ng of total RNA on a Nanostring nCounter system using markers within NS_MM_EXHAUSTION codeset. RCC files were imported into nSolver 4.0 software and analyzed using the default analysis pipeline for normalization, differential expression, and agglomerative clustering.

### Statistical Analysis

GraphPad Prism was used to calculate p values with one-way analysis of variation (ANOVA) with Dunnett's multiple comparison test, unpaired Student's t-test or paired Student's t-test, as indicated in figure legends. Tumor growth curves were analyzed by simple linear regression. Values of p<0.05 were considered significant. p<0.05 values were ranked as: *p<0.05, **p<0.01, ***p<0.001, ****p<0.0001.

### Example 1

### Stat5b* Supports CD8⁺T Cell Engraftment in the Absence of Lymphodepletion

### Materials and Methods

### Vectors

The plasmid vectors pT-effluc-thy1.1 (19) and pCMV-m7pB (20) were described previously. The pT-SB-Stat5b was synthesized by Vector Builder Biosciences (Chicago, IL). All plasmid vectors were confirmed by DNA sequencing. Transfection-grade plasmids were prepared according to the manufacturers protocol using endotoxin-free buffers and ZymoPURE II Plasmid Maxiprep Kits (ZYMO Research, USA).

### Mice

OT1 (C57BL/6-Tg(TeraTcrb)1100Mjb/J) and B6 albino (B6(Cg)-Tyr^{c-2J}/J) mice were obtained from the Jackson Laboratory. All animal experiments were approved by the Medical University of South Carolina (MUSC) Institutional Animal Care and Use Committee and the Division of Laboratory Animal Resources at MUSC maintained all mice.

### Cell Cultures

B16-F10 and LLC-OVA tumor lines were maintained in Dulbecco's Modified Eagle's Medium (DMEM) supplemented with 10% FBS. All tumor lines were determined to be mycoplasma -free in December 2021. OT1 T cells were maintained in RPMI supplemented with 10% FBS, 300 mg/L L-glutamine, 100 units/mL penicillin, 100 μg/mL streptomycin, 1 mM sodium pyruvate, 100 μM NEAA, 1 mM HEPES and 55 μM 2-mercaptoethanol . For OT1 and PMEL T-cell activation and expansion, whole splenocytes from OT1 or PMEL mice were activated with 1 μg/mL OVA 257-264 peptide or 1 μg/mL glycoprotein 100 (gp100) and expanded for 3 days with 200 U/mL rhIL-2 (NCI). T cells were split on day 3 and expanded in rhIL-2 or IL-15 (50 ng/ml) through day 7. Human PBMCs were activated with CD3/CD28 activators and expanded in 500 U rhIL-2 (NCI) and cultures were maintained in RPMI supplemented with 10% FBS, 300 mg/L L-glutamine, 2 mM GlutaMAX, 100 units/mL penicillin, 100 μg/mL streptomycin, 50 μg/mL gentamycin, 25 mM HEPES and 55 μM 2-mercaptoethanol.

### Adoptive Transfer Models

After in vitro expansion, lymphocytes cells were washed briefly in PBS by centrifugation and transfected with the Neon (Life Technologies, Grand Island, NY) transfection system according to the manufacturer's instructions for mouse T cells as described previously (19). In vitro-expanded OT-1 T cells were prepared for adoptive transfer by transfection with 5 μg pCMV-M7PB and 10 μg of pT-effluc-thy1.1 or 10 μg of pT-GFP and 10 μg of pSB-mSTAT5. Prior to adoptive transfer, mice were preconditioned by exposure to 5 Gy of lymphodepleting radiation using a cesium irradiator unless otherwise indicated. T cells were transfected as described above, allowed to recover in complete TCM for 1 h and then adoptively transferred via IP injection. For ex vivo analysis of ff-Luc-Thy1.1 or GFP transferred T cells, mice were sacrificed 3 days after transfer and organs were processed to single-cell suspensions for FACS analysis.

### Antigen Challenge

For primary challenges, mice were injected subcutaneously (s.c.) with 2.5×105 LLC-ova tumor cells as initial OT-1+ T cell luciferase luminescence faded.

### Tumor Models

For adoptive cellular therapy experiments, LLC-ova adenocarcinomas or B16-F10 melanomas were established subcutaneously by injecting 2.5×105 cells into the right flank of male B6(Cg)-Tyr^{c-2J}/J mice and tumor-bearing hosts were irradiated with 5 Gy 24 hours prior to T-cell transfer. After 7 days of tumor growth, 1×10⁷ OT1 or PMEL T cells were transfected using the Neon transfection system prior to infusion in 100 μL phosphate-buffered saline via IP injection into mice. Tumor growth was measured every other day with calipers, and survival was monitored with an experimental endpoint of tumor growth >400 mm². For ex vivo analysis of ff-Luc-Thy1.1 transferred T cells, mice were sacrificed 5 days after transfer and organs were processed to single-cell suspensions for FACS analysis.

### Bioluminescent Imaging

Mice were anesthetized using isoflurane and injected IP with luciferin substrate (Perkin Elmer, Waltham, MA) at standard concentration of 150 mg/kg in PBS. Approximately 10 minutes after luciferase injections, mice were imaged on the AMI-HT. All data shown represent mean luminescence observed by summing dorsal and ventral measurements obtained from identical regions of interest drawn over the trunk and head of each individual mouse.

### Assessment of Serum

Blood was collected in Microvette CD300 Potassium EDTA collection tubes (Sarstedt, Newton, NC) by retroorbital vein bleeding. Serum was measured using the Mouse Cytokine Proinflammatory Focused 10-Plex Discovery Assay (MDF10) (Eve Biotechnologies, Calgary AB, Canada).

### Flow Cytometry

Fluorescent dye-conjugated antibodies were purchased from Biolegend (San Diego, CA), eBioscience (San Diego, CA) or BD Pharmigen (Mountain View, CA) with the following specificities: CD90.1, CD8a, CD44, CD62L, PD-1, Tim-3 and Lag-3. Cell surface antibody staining was done in PBS containing 2% FBS. Antibody-stained cells were analyzed on a CytoFLEX flow cytometer (Beckman Coulter Life Sciences, Indianapolis, IN) and analysis was performed with FlowJo software.

### Statistical Analysis

GraphPad Prism was used to calculate p values with one-way analysis of variation (ANOVA) with Dunnett's multiple comparison test, unpaired Student's t-test or paired Student's t-test, as indicated in figure legends. Values of p<0.05 were considered significant. p<0.05 values were ranked as: *p<0.05, **p<0.01, ***p<0.001, ****p<0.0001.

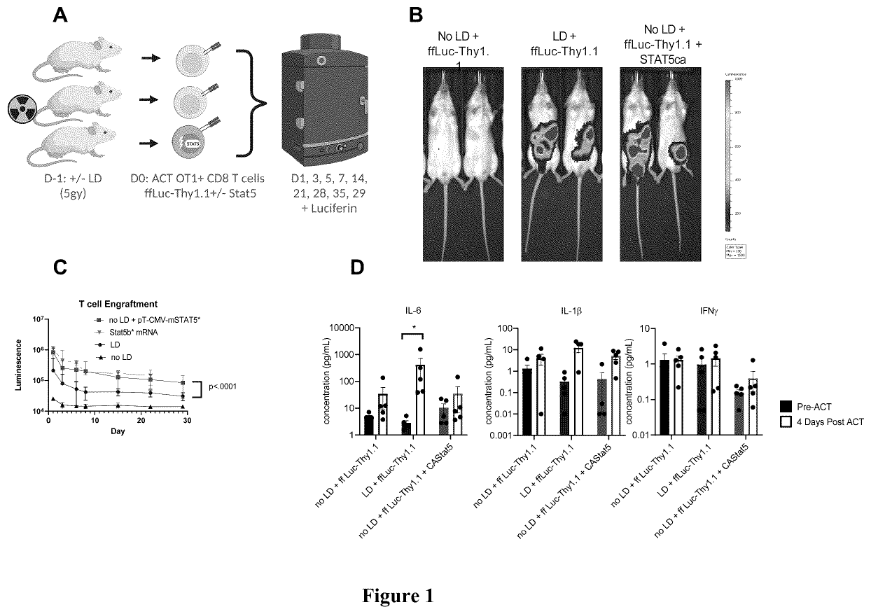

To evaluate the ability of T cells to engraft, the inventors first modified murine OT-1 T cells with the previously described pT-effluc-thy1.1 transposon (21). Luciferase modification enabled the inventors to track the T cells in vivo and quantify engraftment and persistence by measuring luciferase longitudinally. Using this model system, the inventors transiently transfected a plasmid encoding a constitutively active version of murine Stat5b (pCMV-mStat5b*) into our OT-1 T lymphocytes prior to adoptive transfer (FIG. 1a). The inventors hypothesized that this novel engraftment strategy would activate pathways downstream of the γc cytokine receptors to facilitate T cell engraftment without prior lymphodepletion. The inventors observed engraftment of adoptively transferred luciferase positive cells to lymphatic tissues in lymphodepleted recipients, as well as lymphoreplete animals transferred with T cells expressing constitutively active Stat5b* plasmid or mRNA (FIG. 1b). This luciferase signal peaked at 24h followed by luciferase signal decay over time in accordance with expected kinetics in a host without OVA antigen expression in any tissue (FIG. 1c). Interestingly, the inventors noted that cells transfected with pCMV-mStat5b* or Stat5b* mRNA engrafted more efficiently than the cells transferred into lymphodepleted host. This suggests that Stat5b* expression during ACT could result in more efficient engraftment and provide an opportunity to reduce the numbers of cells required to effectively treat patients.

Engrafting T cells without prior lymphodepletion eliminates pre-clinical correlates of CRS one of the most frequent and serious adverse effects of T cell ACT based these therapies is termed cytokine release syndrome (CRS). CRS has been linked to excessive production of IL-6 during T cell engraftment. This excessive IL-6 is not produced by the engrafting CAR-T cells, rather it is produced by recipient myeloid cells in response to production if cytokines like IL1b by the proliferating T cells and as a result of reactive myelopoiesis induced by lymphodepleting regimens. To investigate the differences in cytokine levels before and after ACT in the context of transient Stat5*, serum concentrations were assessed using a mouse cytokine proinflammatory focused 10-plex discovery assay (Eve Technologies) for mice of each treatment group. Consistent with previous observations regarding CRS and cytokines levels noted above, the only cytokine that was observed to increase after ACT was IL-6. Remarkably, the increase in serum IL-6 was lost in the mice that did not undergo lymphodepletion (11). Taken together, this suggests that ACT of CAR-T cells without prior lymphodepletion may represent an opportunity to reduce or eliminate the risk of CRS in patients.

Adoptively transferred T cells maintain functional surveillance of OVA expressing tumor cells long after engraftment.

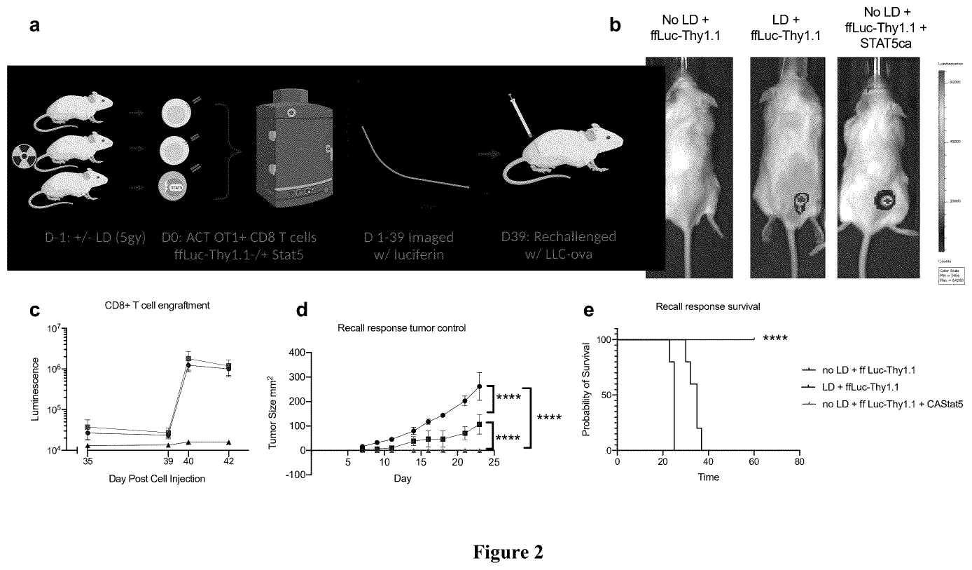

In addition to tracking the persistence of T cells in vivo, luciferase modification allowed the inventors to evaluate their ability to respond to antigen challenge. To this end, as initial luminescence faded, the inventors injected LLC-ova lung cancers subcutaneously into the flanks of each mouse. The inventors observed a rapid response of antigen-specific memory CD8⁺T cells indicated by the increase in bioluminescence at 24h in both the lymphodepleted cohort and the lymphoreplete cohort engrafted with constitutively active Stat5b* plasmid or mRNA (FIG. 2b, c supplement and will add RNA data to figures).

### Example 2

### T Cell Evaluation

To further evaluate the functional fitness of the T cells that were engrafted using transient Stat5* the inventors next evaluated the effector function of the engrafted antigen-specific T cells. As expected, the engrafted T cells provided significant tumor control in the lymphodepleted group, although this group posed a lesser anti-tumor effect than that of the Stat5b* plasmid or mRNA or counterparts. Strikingly, many of the mice engrafted with ACT using Stat5* without lymphodepletion provided complete tumor control and significantly enhanced overall survival in host mice (FIG. 2d, e), implying that this engraftment method yields T cells with functionally superior phenotypes. This prompted investigations into the phenotype of cells generated by the respective modes of engraftment.

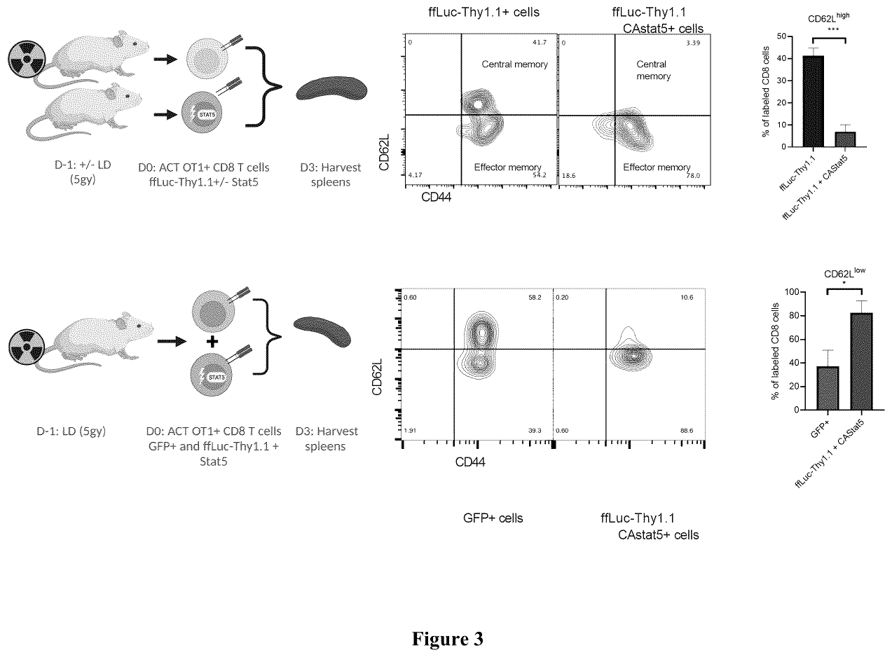

Stat5b* promotes development of a non-canonical effector memory cell phenotype. The inventors examined the phenotype of cells that had undergone adoptive transfer under each condition by collecting cells from spleen, lymph nodes, or tumors. Interestingly, the inventors observed that cells engrafted using the transient Stat5* protocol, failed to upregulate CD62L after adoptive transfer as is observed using conventional lymphodepletion regimens. (FIG. 3) To further investigate if the lymphodepletion or the Stat5* had a dominant effect in suppressing CD62L expression, the inventors conducted a competition assay where mice were lymphodepleted and then engrafted with Stat5* transfected thy1.1 positive cells and sham transfected GFP+ cells. Comparison of CD62L expression between the Stat5* positive and negative cells further supports a model where Stat5* drives dominant alterations in cell phenotype as defined by CD62L.

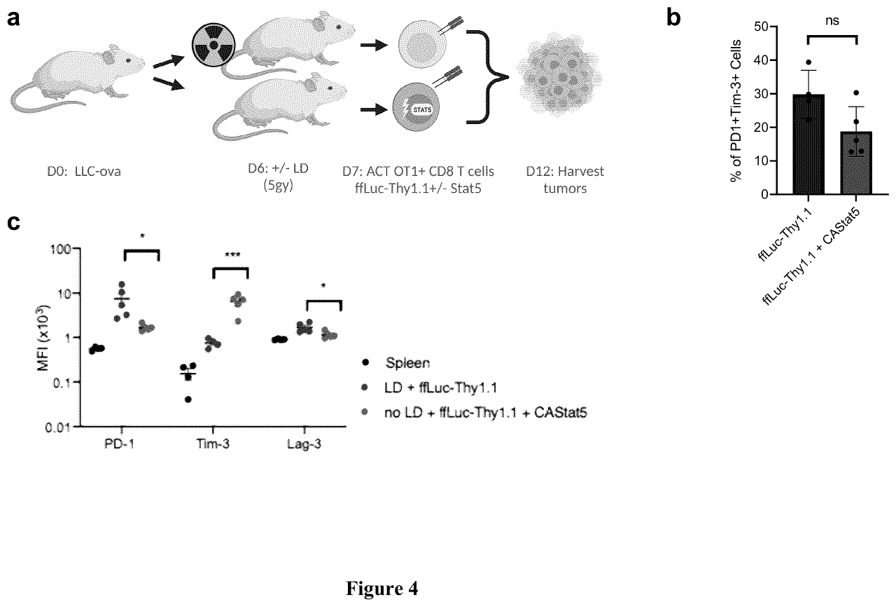

Transient expression of Stat5* during engraftment reduces PD1+Tim3+in solid tumor models. To determine if antitumor function would be altered in Stat5* engrafted mice, the inventors conducted studies to examine the phenotype of adoptively transferred T cells within an immune suppressive tumor model (FIG. 4). Cells were adoptively transferred using conventional lymphodepletion or by transiently expressing Stat5* during engraftment. Tumors were harvested 6 days after ACT and cell phenotype was characterized by flow cytometry. The inventors observed a unique cell phenotype in the Stat5* group where the T cells displayed significant reductions in PD1 expression compared to cells engrafted using conventional lymphodepletion. Consistent with previous observations, Tim3 expression increases as a result of transient Stat5b* expression compared to tumor infiltrating cells engrafted using conventional lymphodepletion. Tim3 has often been identified as a marker of exhaustion when combined with PD1 expression, however the inventors see independent regulation of the two genes in response to Stat5b*. This results in lower numbers of canonically exhausted cells based on expression of both Tim3+ and PD1+ cells in tumors.

### Example 3

### Transient Expression of Stat5b* During Engraftment Promotes Tumor Control

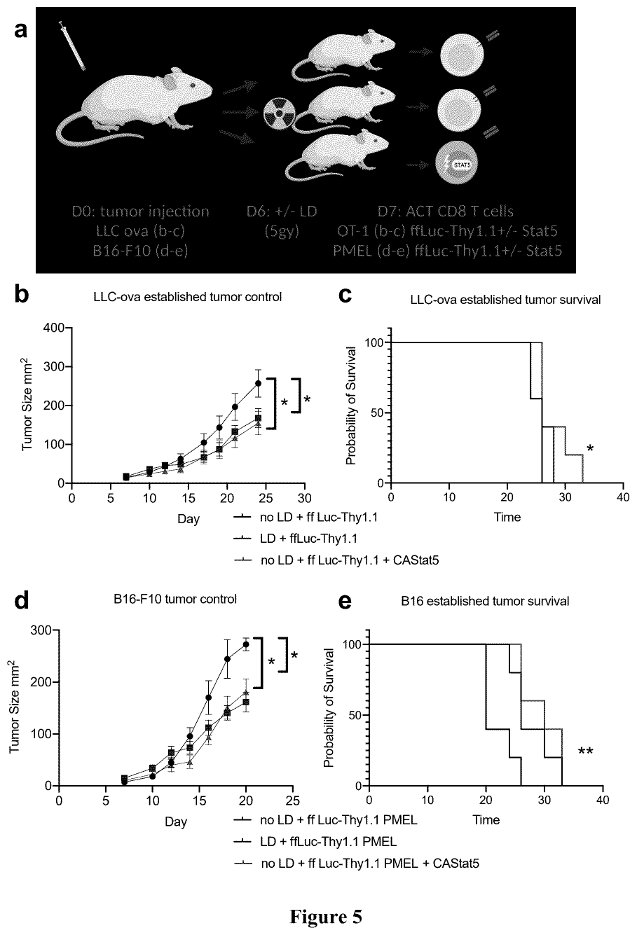

The inventors assessed the functional consequences of Stat5b* activation on tumor control in lymphoreplete hosts. Control luciferase labeled T cells or Stat5b* luciferase labeled T cells were infused into LLC-ova (FIG. 5b, c) or B16-F10 (FIG. 5d, e) tumor-bearing mice. Strikingly, Stat5b* T cells exhibited potent and durable tumor control that posed a similar effect as that of T cell counterparts engrafted through standard lymphodepletion regimens (FIG. 5b, d). As expected, treating lymphoreplete hosts with control T cells abrogated tumor control and diminished survival in both tumor models, supporting the previously described role for lymphodepletion to sustain antitumor immunity of adoptively transferred T cells. In line with these data, survival was substantially enhanced in lymphodeplete mice infused with antigen specific T cells and in lymphoreplete hosts with Stat5b* conditioned T cells compared to respective control T cells in lymphoreplete hosts. Taken together, these data demonstrate Stat5b* is substantial for T cell mediated tumor control in lymphoreplete hosts.

### Example 4

### Cryopreserved CAR-T Cell Product

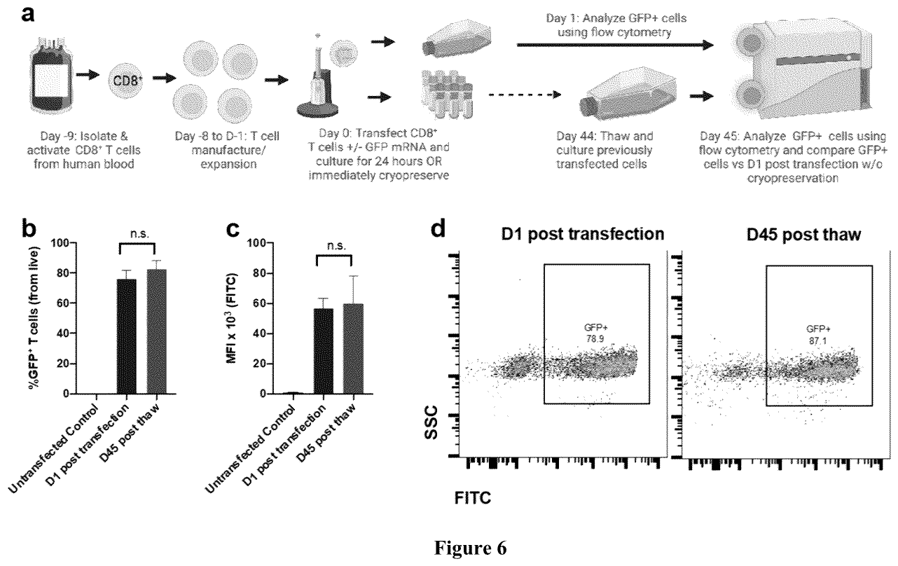

To facilitate point-of-manufacture processing, provided is a novel cryopreserved CAR-T cell product that contains hyper-stable mRNA encoding Stat5* that is ready-to-thaw and infuse into patients including lymphoreplete hosts. FIG. 6 demonstrates stability and efficient expression of mRNA in human CAR-T cells after thawing. FIG. 6 shows experimental schema for isolating human CD8+ T cells from peripheral blood, expansion and transfection of cells. After transfection, cells were cultured for 24 hours or cryopreserved and thawed after 44 days before flow cytometry analysis. FIG. 6 also shows summary data demonstrating the percentage of live, transfected cells that are (b) expressing GFP or (c) the intensity of GFP measured by FITC MFI and (d) representative flow cytometry plots of GFP+ cells one day after transfection compared to after cryopreservation when thawed and analyzed 45d post manufacture.

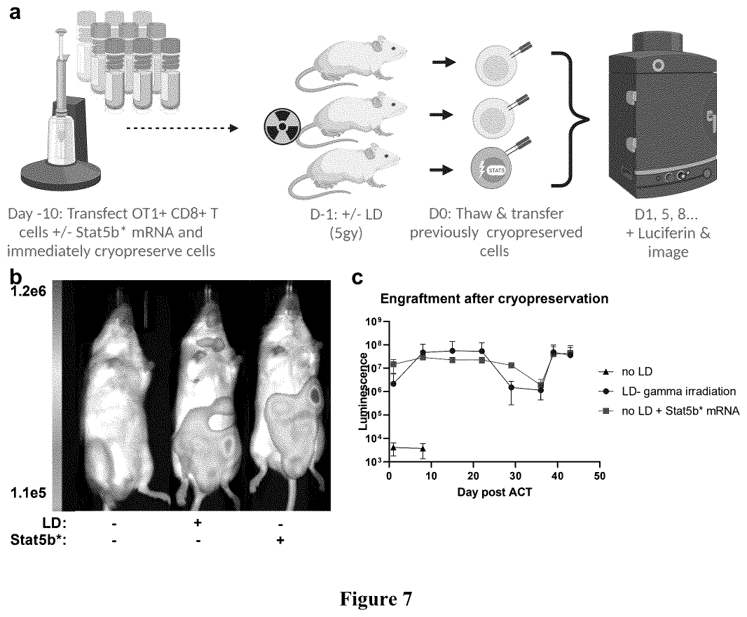

Moreover, FIG. 7 demonstrates the stability and function of our Stat5b* mRNA in mediating engraftment in lymphoreplete animals after thawing cryopreserved cells. Panel a) shows an experimental schematic demonstrating three groups of animals that underwent adoptive cell therapy (ACT) after cells had been transfected with a control mRNA or Stat5b* mRNA and immediately cryopreserved for 10 days, prior to being thawed and transferred into the animals. The experimental groups received ACT after no lymphodepletion, conventional 5 Gy irradiation lymphodepletion, or no lymphodepletion followed by ACT with cells transfected with Stat5b* mRNA. Panel b) shows representative ventral images of luciferase engineered CD8+ T cells 5 days after ACT. Panel c) shows animals were measured (ongoing study) for 8 days post ACT and the intensity of the signal emitted by the luciferase engineered T cells was expressed as luminescence. Quantification of total (dorsal+ventral) luminescence is plotted (n=5 for all groups).

Together, these data support the invention that Stat5b* can be transfected after CAR-T cell manufacture, immediately cryopreserved, and shipped to the point-of-care where the cells are thawed and infused into lymphoreplete patients, obviating the need for lymphodepletion prior to adoptive transfer.

### Example 5

### Gene Expression Changes in Mouse CD8⁺T Cells Transfected with Stat5b*

The inventors believe that adoptive cell therapy could be improved by RNA transfection of a constitutively active Stat5 transcript prior to adoptive transfer. To evaluate this process, the inventors leveraged Stat5b* plasmid DNA or mRNA to transfect OT1⁺CD8⁺T cells and measured gene expression using the nanoString™ (Seattle, WA) platform using the NS_MM_EXHAUSTION CSO code set. The inventors first evaluated overall survival and transfection efficiency of T cells after RNA and DNA electroporation and found that RNA transfection was associated with lower cellular toxicity (FIG. S2a, b) and higher transfection efficiency. The inventors then harvested RNA from cells and analyzed them using an off-the-shelf nanoString™ (Seattle, WA) Immune Exhaustion Panel (NS_MM_EXHAUSTION Panel: Cat# PSTD-M-EXHAUST-12). The results of this analysis clearly indicate that, in CD8⁺T cells, Stat5b* RNA transfection increases expression of genes normally associated with Stat5b activation (Socs genes, Grzmb, Ccr5) while decreasing expression of genes known to be downregulated by Stat5b activation (Il7r, Jak3, Ccr7).

The invention is further described in the following numbered paragraphs:

1. A method for functional engraftment of cells in the absence of lymphodepletion, comprising the step of transient transfection of T cells with a constitutively active STAT5 prior to adoptive transfer.

2. A method for reducing cytokine release syndrome (CRS) in a patient undergoing cancer therapy, comprising the step of administering to said patient T cells transiently transfected with a constitutively active STAT5 prior to adoptive transfer.

3. A method for increasing efficiency of engraftment of cells, comprising the step of transient transfection of T cells with STAT5 prior to adoptive transfer.

4. A method for reducing production of IL-6 in a patient in need thereof, comprising the step of administering to said patient T cells transiently transfected with a constitutively active STAT5 prior to adoptive transfer.

5. A method of producing a cryopreserved T cell product, comprising the steps of:
transiently transfecting T cells with a constitutively active STAT5 prior to adoptive transfer to produce a transfected T cell; and
cryopreserving said transfected T cells.

6. A point-of-use product, comprising the cryopreserved transfected T cell according to paragraph 5.

7. A method for reducing cytokine release syndrome (CRS) in a patient undergoing cancer therapy, comprising the step of administering to said patient the cryopreserved transfected T cell according to paragraph 6 after thawing.

8. A method for reducing production of IL-6 in a patient in need thereof, comprising the step of administering to said patient the cryopreserved transfected T cell according to paragraph 6 after thawing.

### References

1. Lee D W, III, Stetler-Stevenson M, Yuan C M, Shah N N, Delbrook C, Yates B, Zhang H, Zhang L, Kochenderfer J N, Rosenberg S A, Fry T J, Stroncek D, Mackall C L. Long-Term Outcomes Following CD19 CAR T Cell Therapy for B-ALL Are Superior in Patients Receiving a Fludarabine/Cyclophosphamide Preparative Regimen and Post-CAR Hematopoietic Stem Cell Transplantation. Blood. 2016; 128(22):218-. doi:10.1182/blood.V128.22.218.218.
2. Turtle C J, Hanafi L A, Berger C, Hudecek M, Pender B, Robinson E, Hawkins R, Chaney C, Cherian S, Chen X, Soma L, Wood B, Li D, Heimfeld S, Riddell S R, Maloney D G. Immunotherapy of non-Hodgkin's lymphoma with a defined ratio of CD8+ and CD4+CD19-specific chimeric antigen receptor-modified T cells. Sci Transl Med. 2016; 8(355):355ra116. Epub 2016 Sep. 9. doi:10.1126/scitranslmed.aaf8621. PubMed PMID: 27605551; PMCID: PMC 5045301.
3. Gattinoni L, Finkelstein S E, Klebanoff C A, Antony P A, Palmer D C, Spiess P J, Hwang L N, Yu Z, Wrzesinski C, Heimann D M, Surh C D, Rosenberg S A, Restifo N P. Removal of homeostatic cytokine sinks by lymphodepletion enhances the efficacy of adoptively transferred tumor-specific CD8+ T cells. J Exp Med. 2005; 202(7):907-12. Epub 2005 Oct. 6. doi:10.1084/jem.20050732. PubMed PMID: 16203864; PMCID: PMC1397916.
4. Antony P A, Restifo N P. CD4+CD25+T regulatory cells, immunotherapy of cancer, and interleukin-2. J Immunother. 2005; 28(2):120-8. Epub 2005 Feb. 24. doi:10.1097/01.cji.0000155049.26787.45. PubMed PMID: 15725955; PMCID: PMC 1242172.
5. Antony P A, Piccirillo C A, Akpinarli A, Finkelstein S E, Speiss P J, Surman D R, Palmer D C, Chan C C, Klebanoff C A, Overwijk W W, Rosenberg S A, Restifo N P. CD8+T cell immunity against a tumor/self-antigen is augmented by CD4+T helper cells and hindered by naturally occurring T regulatory cells. J Immunol. 2005; 174(5):2591-601. Epub 2005 Feb. 25. doi:10.4049/jimmunol.174.5.2591. PubMed PMID: 15728465; PMCID: PMC 1403291.
6. Bronte V, Apolloni E, Cabrelle A, Ronca R, Serafini P, Zamboni P, Restifo NP, Zanovello P. Identification of a CD11b(+)/Gr-1(+)/CD31(+) myeloid progenitor capable of activating or suppressing CD8(+) T cells. Blood. 2000; 96(12):3838-46. Epub 2000 Nov. 23. PubMed PMID: 11090068; PMCID: PMC2734459.
7. Dudley M E, Yang J C, Sherry R, Hughes M S, Royal R, Kammula U, Robbins P F, Huang J, Citrin D E, Leitman S F, Wunderlich J, Restifo N P, Thomasian A, Downey S G, Smith F O, Klapper J, Morton K, Laurencot C, White D E, Rosenberg S A. Adoptive cell therapy for patients with metastatic melanoma: evaluation of intensive myeloablative chemoradiation preparative regimens. J Clin Oncol. 2008; 26(32):5233-9. Epub 2008 Sep. 24. doi:10.1200/jco.2008.16.5449. PubMed PMID: 18809613; PMCID: PMC2652090.
8. Muranski P, Boni A, Wrzesinski C, Citrin D E, Rosenberg S A, Childs R, Restifo N P. Increased intensity lymphodepletion and adoptive immunotherapy—how far can we go? Nat Clin Pract Oncol. 2006; 3(12):668-81. Epub 2006 Dec. 2. doi:10.1038/ncponc0666. PubMed PMID: 17139318; PMCID: PMC1773008.
9. Chen C I, Abraham R, Tsang R, Crump M, Keating A, Stewart A K. Radiation-associated pneumonitis following autologous stem cell transplantation: predictive factors, disease characteristics and treatment outcomes. Bone Marrow Transplant. 2001; 27(2):177-82. Epub 2001 Apr. 3. doi:10.1038/sj.bmt.1702771. PubMed PMID: 11281387.
10. Schubert M L, Schmitt M, Wang L, Ramos C A, Jordan K, Muller-Tidow C, Dreger P. Side-effect management of chimeric antigen receptor (CAR) T-cell therapy. Ann Oncol. 2021; 32(1):34-48. doi:10.1016/j.annonc.2020.10.478. PubMed PMID: 33098993.
11. Innamarato P, Kodumudi K, Asby S, Schachner B, Hall M, Mackay A, Wiener D, Beatty M, Nagle L, Creelan B C, Sarnaik A A, Pilon-Thomas S. Reactive Myelopoiesis Triggered by Lymphodepleting Chemotherapy Limits the Efficacy of Adoptive T Cell Therapy. Mol Ther. 2020; 28(10):2252-70. doi:10.1016/j.ymthe.2020.06.025. PubMed PMID: 32615068; PMCID: PMC 7544980.
12. Dudley M E, Wunderlich J R, Yang J C, Hwu P, Schwartzentruber D J, Topalian S L, Sherry R M, Marincola F M, Leitman S F, Seipp C A, Rogers-Freezer L, Morton K E, Nahvi A, Mavroukakis S A, White D E, Rosenberg S A. A phase I study of nonmyeloablative chemotherapy and adoptive transfer of autologous tumor antigen-specific T lymphocytes in patients with metastatic melanoma. J Immunother. 2002; 25(3):243-51. Epub 2002 May 10. doi:10.1097/01.Cji.0000016820.36510.89. PubMed PMID: 12000866; PMCID: PMC 2413438.
13. Dudley M E, Wunderlich J R, Robbins P F, Yang J C, Hwu P, Schwartzentruber D J, Topalian S L, Sherry R, Restifo N P, Hubicki A M, Robinson M R, Raffeld M, Duray P, Seipp C A, Rogers-Freezer L, Morton K E, Mavroukakis S A, White D E, Rosenberg S A. Cancer Regression and Autoimmunity in Patients After Clonal Repopulation with Antitumor Lymphocytes. Science. 2002; 298(5594):850-4. doi:doi:10.1126/science.1076514.
14. Ramos C A, Rouce R, Robertson C S, Reyna A, Narala N, Vyas G, Mehta B, Zhang H, Dakhova O, Carrum G, Kamble R T, Gee A P, Mei Z, Wu M-F, Liu H, Grilley B, Rooney C M, Heslop H E, Brenner M K, Savoldo B, Dotti G. <em>ln&#xa0;Vivo</em> Fate and Activity of Second-versus Third-Generation CD19-Specific CAR-T Cells in B Cell Non-Hodgkin&#x2019;s Lymphomas. Molecular Therapy. 2018; 26(12):2727-37. doi:10.1016/j.ymthe.2018.09.009.
15. Rochman Y, Spolski R, Leonard W J. New insights into the regulation of T cells by gamma(c) family cytokines. Nat Rev Immunol. 2009; 9(7):480-90. Epub 2009 Jun. 23. doi:10.1038/nri2580. PubMed PMID: 19543225; PMCID: PMC 2814538.
16. Grange M, Buferne M, Verdeil G, Leserman L, Schmitt-Verhulst A-M, Auphan-Anezin N. Activated STAT5 Promotes Long-Lived Cytotoxic CD8<sup>+</sup> T Cells That Induce Regression of Autochthonous Melanoma. Cancer Research. 2012; 72(1):76-87. doi:10.1158/0008-5472.Can-11-2187.
17. Gouilleux F, Wakao H, Mundt M, Groner B. Prolactin induces phosphorylation of Tyr694 of Stat5 (MGF), a prerequisite for DNA binding and induction of transcription. Embo j. 1994; 13(18):4361-9. Epub 1994 Sep. 15. PubMed PMID: 7925280; PMCID: PMC 395363.
18. Onishi M, Nosaka T, Misawa K, Mui A L, Gorman D, McMahon M, Miyajima A, Kitamura T. Identification and characterization of a constitutively active STAT5 mutant that promotes cell proliferation. Mol Cell Biol. 1998; 18(7):3871-9. Epub 1998 Jun. 25. doi:10.1128/mcb.18.7.3871. PubMed PMID: 9632771; PMCID: PMC 108971.
19. Galvan D L, O'Neil R T, Foster A E, Huye L, Bear A, Rooney C M, Wilson M H. Anti-Tumor Effects after Adoptive Transfer of IL-12 Transposon-Modified Murine Splenocytes in the OT-I-Melanoma Mouse Model. PLoS One. 2015; 10(10):e0140744. Epub 2015 Oct. 17. doi:10.1371/journal.pone.0140744. PubMed PMID: 26473608; PMCID: PMC 4608718.
20. Doherty J E, Huye L E, Yusa K, Zhou L, Craig N L, Wilson M H. Hyperactive piggyBac gene transfer in human cells and in vivo. Hum Gene Ther. 2012; 23(3):311-20. Epub 2011 Oct. 14. doi:10.1089/hum.2011.138. PubMed PMID: 21992617; PMCID: PMC 3300075.
21. O'Neil R T, Saha S, Veach R A, Welch R C, Woodard L E, Rooney C M, Wilson M H. Transposon-modified antigen-specific T lymphocytes for sustained therapeutic protein delivery in vivo. Nature Communications. 2018; 9(1):1325. doi:10.1038/s41467-018-03787-8.

It is to be understood that the invention is not limited to the particular embodiments of the invention described above, as variations of the particular embodiments may be made and still fall within the scope of the appended claims.

1. A method for functional engraftment of cells in the absence of lymphodepletion, comprising the step of transient transfection of T cells with a constitutively active STAT5 prior to adoptive transfer.

2. A method for reducing cytokine release syndrome (CRS) in a patient undergoing cancer therapy, comprising the step of administering to said patient T cells transiently transfected with a constitutively active STAT5 prior to adoptive transfer.

3. A method for increasing efficiency of engraftment of cells, comprising the step of transient transfection of T cells with STAT5 prior to adoptive transfer.

4. A method for reducing production of IL-6 in a patient in need thereof, comprising the step of administering to said patient T cells transiently transfected with a constitutively active STAT5 prior to adoptive transfer.

5. A method of producing a cryopreserved T cell product, comprising the steps of:
transiently transfecting T cells with a constitutively active STAT5 prior to adoptive transfer to produce a transfected T cell; and
cryopreserving said transfected T cells.

6. A point-of-use product, comprising the cryopreserved transfected T cell according to claim 5.

7. A method for reducing cytokine release syndrome (CRS) in a patient undergoing cancer therapy, comprising the step of administering to said patient the cryopreserved transfected T cell according to claim 6 after thawing.

8. A method for reducing production of IL-6 in a patient in need thereof, comprising the step of administering to said patient the cryopreserved transfected T cell according to claim 6 after thawing.
